(19) United States
(12) Patent Application Publication
(10) Pub. No.: US 2021/0121201 A1
(43) Pub. Date: Apr. 29, 2021

(54) INTRAOSSEOUS ACCESS DEVICE AND METHOD TO ACCESS BONE MARROW

(71) Applicant: TELEFLEX LIFE SCIENCES LIMITED, Valletta (MT)

(72) Inventors: Morgan TIERNEY, Tullamore, Offaly (IE); Alex FOMINAS, Athlone, Co. Roscommon (IE)

(21) Appl. No.: 17/054,385

(22) PCT Filed: May 10, 2019
(86) PCT No.: PCT/IB19/53900

§ 371 Date: Nov. 10, 2020

Related U.S. Application Data
(60) Provisional application No. 62/670,691, filed on May 11, 2018.

(51) Int. Cl. A61B 17/34; A61B 10/02

(52) U.S. Cl. CPC A61B 17/3472; A61B 17/3417; A61B 10/025; A61B 10/0275; A61B 17/3494

(57) ABSTRACT

Penetrator assemblies operable to provide access to an intraosseous space are disclosed. The penetrator assembly may include a flexible outer penetrator having a longitudinal bore and a distal end operable to penetrate bone and associated bone marrow. The penetrator assembly may also include a rigid inner penetrator including a distal end operable to penetrate bone and associated bone marrow. A hub having a distal end is connected to a proximal end of the flexible outer penetrator. A connector having a distal end is connected to a proximal end of the rigid inner penetrator. A proximal end of the connector can releasably engage a driver. The longitudinal bore of the flexible outer penetrator can removably receive the rigid inner penetrator to prevent or minimize the flexible outer penetrator from bending during an insertion procedure. The flexible outer penetrator can bend after removal of the rigid inner penetrator from the longitudinal bore.

### CROSS-REFERENCE TO RELATED APPLICATIONS

This application claims the benefit of priority to U.S. Provisional Patent Application No. 62/670,691, filed May 11, 2018, the disclosure of which is incorporated herein by reference in its entirety.

### TECHNICAL FIELD

The present disclosure generally relates to a medical apparatus for accessing an intraosseous space, and more specifically, to an intraosseous device and method for penetrating a bone and accessing associated bone marrow for medical procedures.

### BACKGROUND

An important element for treating patients experiencing a life-threatening emergency is the rapid establishment of an intravenous (IV) line in order to administer drugs and fluids directly into the circulatory system. Whether in an ambulance by paramedics, or in an emergency room by emergency specialists, the goal is the same—to start an IV in order to administer life-saving drugs and fluids. To a large degree, the ability to successfully treat such critical emergencies is dependent on the skill and luck of the operator in accomplishing vascular access. While it is relatively easy to start an IV on some patients, doctors, nurses and paramedics often experience great difficulty establishing IV access in many patients. These patients are probed repeatedly with sharp needles in an attempt to solve this problem and may require an invasive procedure to finally establish an intravenous route. A further complicating factor in achieving IV access occurs “in the field,” e.g. at the scene of an accident or during ambulance transport where it is difficult to see the target and excessive motion makes accessing the venous system very difficult.

In the case of patients with chronic disease or the elderly, the availability of easily-accessible veins may be depleted. Other patients may have no available IV sites due to anatomical scarcity of peripheral veins, obesity, extreme dehydration or previous IV drug use. For these patients, finding a suitable site for administering lifesaving drugs becomes a difficult and frustrating task. Many patients with life-threatening emergencies have died of ensuing complications because access to the vascular system with life-saving IV therapy was delayed or simply not possible.

For such patients, an alternative approach which uses the intraosseous (IO) space to provide a direct conduit to a patient's vascular system is an attractive alternate route to administer IV drugs and fluids. Drugs administered intraosseously enter a patient's blood circulation system as rapidly as they do when given intravenously. However, proper placement of the intraosseous needle in a target site is critical. If a user attempts to insert the needle in the wrong place, the bone might be too thick and therefore difficult for the needle to penetrate. Alternatively, in other places the bone might be too thin, and thus the needle could completely penetrate through the entire bone, thus passing the intraosseous space. Furthermore, placing the needle at an angle in the bone, where the needle is not substantially perpendicular to the chest of the patient, may lead to the needle breaking or other complications.

Additionally, a conventional rigid cannula inserted into the bone may unintentionally become dislodged by bumping it or upon movement of the patient. For instance, such a conventional rigid cannula inserted into a humerus of a patient may inadvertently become dislodged upon movement of the patient's arm, i.e., when a patient raises their arm above their head, or when the patient accidentally knocks the inserted cannula out of position. Therefore, there is a need for a penetrator assembly having flexible cannula to prevent or decrease the chance of it unintentionally dislodging from a patient's bone at a target site.

### SUMMARY

The foregoing needs are met by implementations of a penetrator assembly according to the present disclosure, in which the penetrator assembly is operable to provide access to an intraosseous space. According to one aspect of the disclosure, the penetrator assembly comprises a flexible outer penetrator including a longitudinal bore and a distal end operable to penetrate bone and associated bone marrow; a rigid inner penetrator including a distal end operable to penetrate bone and associated bone marrow; a hub having a distal end connected to a proximal end of the flexible outer penetrator; a connector having a distal end connected to a proximal end of the rigid inner penetrator and a proximal end configured to releasably engage a driver; and the longitudinal bore of the flexible outer penetrator configured to removably receive the rigid inner penetrator to prevent or minimize the flexible outer penetrator from bending during an insertion procedure, and the flexible outer penetrator configured to bend after removal of the rigid inner penetrator from the longitudinal bore.

According to another aspect of the disclosure, the penetrator assembly further comprises a depth control collar connected to the outer penetrator adjacent to the distal end of the outer penetrator, the depth control collar comprising a distal end operable to contact the bone for preventing further penetration of the inner and outer penetrators into the intraosseous space.

According to another aspect of the disclosure, the penetrator assembly further comprises a distal cutting sleeve fixed to the distal end of the outer penetrator, the distal cutting sleeve operable to penetrate bone and associated bone marrow.

According to another aspect of the disclosure, the flexible outer penetrator includes a plurality of slits operable to allow the outer penetrator to bend when the rigid inner penetrator is removed from the longitudinal bore of the flexible outer penetrator.

According to another aspect of the disclosure, the plurality of slits are provided along an intermediate section of the flexible outer penetrator, the intermediate section located between the distal and proximal ends of the flexible outer penetrator.

According to another aspect of the disclosure, the plurality of slits are arranged in a pattern along the length of the intermediate section of the flexible outer penetrator.

According to another aspect of the disclosure, the pattern of the plurality of slits includes three cut lines per pitch.

According to another aspect of the disclosure, each slit in the pattern of the plurality of slits increments rotationally by 90 degrees.

According to another aspect of the disclosure, at least one of the plurality of slits is an elongated cut in a wall of the flexible outer penetrator.

According to another aspect of the disclosure, at least one of the plurality of slits is a perforation in a wall of the flexible outer penetrator.

According to another aspect of the disclosure, at least one of the plurality of slits is a notch or groove in a wall of the flexible outer penetrator.

According to another aspect of the disclosure, the plurality of slits are formed by a laser.

According to another aspect of the disclosure, the plurality of slits are formed by chemical etching.

According to another aspect of the disclosure, the plurality of slits are formed by water blasting.

According to another aspect of the disclosure, a covering sleeve may be attached to the flexible outer penetrator, the covering sleeve operable to prevent or minimize leakage of material from the flexible outer penetrator.

According to another aspect of the disclosure, the covering sleeve is configured to cover the intermediate section of the flexible outer penetrator, the covering sleeve operable to prevent or minimize leakage of material from the plurality of slits.

According to another aspect of the disclosure, the covering sleeve is operable to bend when bending the flexible outer penetrator.

According to another aspect of the disclosure, the covering sleeve is operable to prevent or reduce leakage of material passing through the outer penetrator at a high pressure.

According to another aspect of the disclosure, the covering sleeve is a heat-shrink tubing.

According to another aspect of the disclosure, the covering sleeve comprises a polymer.

According to another aspect of the disclosure, the covering sleeve comprises fluorinated ethylene propylene.

According to another aspect of the disclosure, the covering sleeve is transparent.

According to another aspect of the disclosure, the covering sleeve is a helical hollow strand tubing.

According to another aspect of the disclosure, an outer diameter of the covering sleeve is less than or equal to the size of an outer diameter of the collar.

According to another aspect of the disclosure, an outer diameter of the covering sleeve is less than or equal to the size of an outer diameter of the distal cutting sleeve.

According to another aspect of the disclosure, the distal end of the connector is configured to releasably engage the proximal end of the hub.

According to another aspect of the disclosure, the distal end of the connector comprises a female threaded portion and the proximal end of the hub comprises a male threaded portion, the male and female threaded portions operable to threadedly engage the hub to the connector.

According to another aspect of the disclosure, the connector comprises a receptacle configured to receive a drive shaft of a driver.

According to another aspect of the disclosure, the receptacle is further configured to releasably engage the drive shaft of the driver.

According to another aspect of the disclosure, the receptacle comprises a generally tapered configuration operable to releasably fittingly engage with a tapered portion of the drive shaft of the driver.

According to another aspect of the disclosure, the receptacle further comprises a magnetic disk operable to releasably engage a magnetic portion of the drive shaft of the driver.

According to another aspect of the disclosure, the flexible outer penetrator is a cannula.

According to another aspect of the disclosure, the rigid inner penetrator is a stylet.

According to another aspect of the disclosure, a penetrator assembly operable to provide access to an intraosseous space comprises a fluted drill tip operable to penetrate bone and associated bone marrow; a flexible cannula including a longitudinal bore and a distal end connected to the fluted drill tip; a rigid stylet configured to be releasably received within the longitudinal bore of the flexible cannula, the rigid stylet operable to prevent or minimize the flexible cannula from bending during an insertion procedure when the rigid stylet is received within the longitudinal bore of the flexible cannula, and the flexible cannula configured to bend after removal of the rigid stylet from the longitudinal bore; a hub having a distal end connected to a proximal end of the flexible cannula; and a connector having a distal end connected to a proximal end of the rigid stylet, and a proximal end configured to releasably engage a driver.

According to another aspect of the disclosure, the fluted drill tip comprises a head portion, a body portion, and a cutting flute extending along both the head and body portions, the cutting flute defining a channel operable to allows for blood and/or bone marrow samples to be aspirated from the intraosseous space or for medication to be delivered to the intraosseous space.

According to another aspect of the disclosure, the flexible cannula includes a plurality of slits operable to allow the cannula to bend when the rigid stylet is removed from the longitudinal bore of the cannula.

According to another aspect of the disclosure, the plurality of slits are provided along an intermediate section of the flexible cannula, the intermediate section located between the distal and proximal ends of the flexible cannula.

According to another aspect of the disclosure, the plurality of slits are arranged in a pattern along the length of the intermediate section of the flexible cannula.

According to another aspect of the disclosure, the pattern of the plurality of slits includes three cut lines per pitch.

According to another aspect of the disclosure, each slit in the pattern of the plurality of slits increments rotationally by 90 degrees.

According to another aspect of the disclosure, at least one of the plurality of slits is an elongated cut in a wall of the flexible cannula.

According to another aspect of the disclosure, at least one of the plurality of slits is a perforation in a wall of the flexible cannula.

According to another aspect of the disclosure, at least one of the plurality of slits is a notch or groove in a wall of the flexible cannula.

According to another aspect of the disclosure, the plurality of slits are formed by a laser.

According to another aspect of the disclosure, the plurality of slits are formed by chemical etching.

According to another aspect of the disclosure, the plurality of slits are formed by water blasting.

According to another aspect of the disclosure, a covering sleeve may be attached to the flexible cannula, the covering sleeve operable to prevent or minimize leakage of material from the flexible cannula.

According to another aspect of the disclosure, the covering sleeve is configured to cover the intermediate section of the flexible cannula, the covering sleeve operable to prevent or minimize leakage of material from the plurality of slits.

According to another aspect of the disclosure, the covering sleeve is operable to bend when bending the flexible cannula.

According to another aspect of the disclosure, the covering sleeve is operable to prevent or reduce leakage of material passing through the cannula at a high pressure.

According to another aspect of the disclosure, the covering sleeve is a heat-shrink tubing.

According to another aspect of the disclosure, the covering sleeve comprises a polymer.

According to another aspect of the disclosure, the covering sleeve comprises fluorinated ethylene propylene.

According to another aspect of the disclosure, the covering sleeve is transparent.

According to another aspect of the disclosure, the covering sleeve is a helical hollow strand tubing.

According to another aspect of the disclosure, the covering sleeve has an outer diameter that is less than or equal to the size of an outer diameter of the head portion of the fluted cutting tip.

According to another aspect of the disclosure, the distal end of the connector is configured to releasably engage the proximal end of the hub.

According to another aspect of the disclosure, the distal end of the connector comprises a female threaded portion and the proximal end of the hub comprises a male threaded portion, the male and female threaded portions operable to threadedly engage the hub to the connector.

According to another aspect of the disclosure, the connector comprises a receptacle configured to receive a drive shaft of a driver.

According to another aspect of the disclosure, the receptacle is further configured to releasably engage the drive shaft of the driver.

According to another aspect of the disclosure, the receptacle comprises a generally tapered configuration operable to releasably fittingly engage with a tapered portion of the drive shaft of the driver.

According to another aspect of the disclosure, the receptacle further comprises a magnetic disk operable to releasably engage a magnetic portion of the drive shaft of the driver.

According to another aspect of the disclosure, a penetrator assembly operable to provide access to an intraosseous space comprises: a rigid stylet having a distal end operable to penetrate bone and associated bone marrow; a cannula having a distal end operable to penetrate bone and associated bone marrow, and a longitudinal bore configured to receive a portion of the rigid stylet; a flexible tube attached to an exterior surface of a portion of the cannula for providing fluid communication between the cannula and a hub, the flexible tube configured to receive the portion of the rigid stylet; the rigid stylet configured to support the flexible tube during an insertion procedure.

According to another aspect of the disclosure, the cannula further comprises a proximal end defining a female lock portion.

According to another aspect of the disclosure, the rigid stylet further comprises a key section having a distal end defining a male key portion adapted to mate with the female lock portion at the proximal end of the cannula for orienting the distal end of the cannula relative to the distal end of the stylet to form a cutting tip operable to penetrate bone and associated bone marrow.

According to another aspect of the disclosure, the flexible tube is transparent.

There has thus been outlined certain aspects of the disclosure in order that the detailed description thereof may be better understood, and in order that the present contribution to the art may be better appreciated. There are additional implementations of the disclosure that will be described below and which form the subject matter of the claims appended hereto.

In this respect, before explaining at least one aspect of the penetrator assembly in detail, it is to be understood that the penetrator assembly is not limited in its application to the details of construction and to the arrangements of the components set forth in the following description or illustrated in the drawings. The penetrator assembly is capable of aspects in addition to those described, and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods, and systems for carrying out the several purposes of the penetrator assembly. It is important, therefore, that the claims be regarded as including such equivalent constructions insofar as they do not depart from the spirit and scope of the disclosure.

### BRIEF DESCRIPTION OF THE DRAWINGS

In order that the disclosure may be readily understood, aspects of the intraosseous (IO) access device are illustrated by way of examples in the accompanying drawings, in which like parts are referred to with like reference numerals throughout.

FIG. 1 illustrates a perspective view of an intraosseous needle set according to the present disclosure.

FIG. 2 illustrates an exploded perspective view of the intraosseous needle set of FIG. 1.

FIG. 3 illustrates a side elevation cross-sectional view of the intraosseous needle set of FIG. 1.

FIG. 4 illustrates a side elevation cross-sectional view of the intraosseous needle set of FIG. 1, with the inner penetrator separated from the outer penetrator.

FIG. 5 illustrates a tip portion of the intraosseous needle set of FIG. 1.

FIG. 6A illustrates a top view of a tip portion of an inner penetrator of the intraosseous needle set of FIG. 1.

FIG. 6B illustrates a side view of a tip portion of an inner penetrator of the intraosseous needle set of FIG. 1.

FIG. 7A illustrates a side view of an outer penetrator of the intraosseous needle set of FIG. 1.

FIG. 7B illustrates a top view of an outer penetrator of the intraosseous needle set of FIG. 1.

FIG. 7C illustrates a tip portion of the needle set of FIG. 1 according to another aspect of the present disclosure.

FIG. 7D illustrates a portion of the outer penetrator of the needle set of FIG. 1 according to another aspect of the present disclosure.

FIG. 7E illustrates a recovery shape of the outer penetrator of FIG. 7D.

FIG. 7F illustrates a tip portion of the needle set of FIG. 1 according to another aspect of the present disclosure.

FIG. 7G illustrates a portion of the outer penetrator of the needle set of FIG. 1 according to another aspect of the present disclosure.

FIG. 7H illustrates a recovery shape of the outer penetrator of FIG. 7G.

FIG. 7I illustrates a tip portion of the needle set of FIG. 1 according to another aspect of the present disclosure.

FIG. 7J illustrates a portion of the outer penetrator of the needle set of FIG. 1 according to another aspect of the present disclosure.

FIG. 7K illustrates a recovery shape of the outer penetrator of FIG. 7J.

FIG. 7L illustrates a tip portion of the needle set of FIG. 1 according to another aspect of the present disclosure.

FIG. 7M illustrates a portion of the outer penetrator of the needle set of FIG. 1 according to another aspect of the present disclosure.

FIG. 7N illustrates a recovery shape of the outer penetrator of FIG. 7M.

FIG. 7O illustrates a tip portion of the needle set of FIG. 1 according to another aspect of the present disclosure.

FIG. 7P illustrates a portion of the outer penetrator of the needle set of FIG. 1 according to another aspect of the present disclosure.

FIG. 7Q illustrates a recovery shape of the outer penetrator of FIG. 7P.

FIG. 8 illustrates a rear perspective view of a connector of the intraosseous needle set of FIG. 1.

FIG. 9 illustrates a perspective view of an intraosseous needle set having a depth control collar according to the present disclosure.

FIG. 10 illustrates an exploded perspective view of the intraosseous needle set of FIG. 9.

FIG. 11 illustrates a side elevation cross-sectional view of the intraosseous needle set of FIG. 9.

FIG. 12 illustrates a side elevation cross-sectional view of the intraosseous needle set of FIG. 9, with the inner penetrator separated from the outer penetrator.

FIG. 13 illustrates a perspective view of an intraosseous needle set having a distal cutting sleeve according to the present disclosure.

FIG. 14 illustrates an exploded perspective view of the intraosseous needle set of FIG. 13.

FIG. 15 illustrates a side elevation cross-sectional view of the intraosseous needle set of FIG. 13.

FIG. 16 illustrates a side elevation cross-sectional view of the intraosseous needle set of FIG. 13, with the inner penetrator separated from the outer penetrator.

FIG. 17 illustrates a tip portion of the intraosseous needle set of FIG. 13.

FIG. 18A illustrates a side view of the distal cutting sleeve according to the present disclosure.

FIG. 18B illustrates a top view of the distal cutting sleeve according to the present disclosure.

FIG. 19 illustrates a perspective view of an intraosseous needle set having a fluted cutting drill tip according to the present disclosure.

FIG. 20 illustrates an exploded perspective view of the intraosseous needle set of FIG. 19.

FIG. 21 illustrates a side elevation cross-sectional view of the intraosseous needle set of FIG. 19.

FIG. 22 illustrates a front elevation view of the fluted cutting drill tip according to the present disclosure.

FIG. 23 illustrates a partial cross-sectional view of a portion of the intraosseous needle set of FIG. 19.

FIG. 24 illustrates a side elevation view of the fluted cutting drill tip according to the present disclosure.

FIG. 25A illustrates an intraosseous needle set having a key system according to an aspect of the present disclosure.

FIG. 25B illustrates a tip portion of the intraosseous needle set of FIG. 25A.

FIG. 25C illustrates a manual driver connected to an intraosseous needle set having a key system according to an aspect of the present disclosure.

FIG. 26 illustrates an intraosseous needle set according to an aspect of the present disclosure, with a rigid inner penetrator separated from a flexible outer penetrator.

FIG. 27 illustrates the outer penetrator of the flexible intraosseous needle set of FIG. 26 in a bent configuration.

### DETAILED DESCRIPTION

The present disclosure provides an intraosseous (IO) device and method for penetrating a bone and accessing associated bone marrow for medical procedures, such as aspiration and biopsy of the bone marrow. The term “intraosseous (IO) device” may be used in this application to include, but is not limited to, any hollow needle, hollow drill bit, penetrator assembly, bone penetrator, catheter, cannula, trocar, stylet, inner penetrator, outer penetrator, IO needle, biopsy needle, aspiration needle, IO needle set, biopsy needle set, or aspiration needle set operable to provide access to an intraosseous space or interior portions of a bone. However, a wide variety of other IO devices may be formed in accordance with one or more teachings of the present disclosure. Such IO devices may be formed, at least in part, from metal alloys such as 304 stainless steel and other biocompatible materials associated with needles and similar medical devices.

The term “fluid” may be used in this application to include liquids such as, but not limited to, blood, water, saline solutions, IV solutions, plasma or any mixture of liquids, particulate matter, dissolved medication and/or drugs associated with biopsy or aspiration of bone marrow or communication of fluids with bone marrow or other target sites. The term “fluid” may also be used in this patent application to include any body fluids and/or liquids containing particulate matter such as bone marrow and/or cells which may be withdrawn from a target area.

The terms “harvest” and “harvesting” may be used in this application to include bone and/or bone marrow biopsy and bone marrow aspiration. Bone and/or bone marrow biopsy (sometimes referred to as “needle biopsy”) may be generally described as removing a relatively small piece or specimen of bone and/or bone marrow from a selected target area for biopsy purposes. Bone marrow aspiration (sometimes referred to as “bone marrow sampling”) may be generally described as removing larger quantities of bone marrow from a selected target area. Relatively large quantities of bone marrow may be used for diagnostic, transplantation and/or research purposes. For example, some stem cell research techniques may require relatively large quantities of bone marrow.

The terms “insertion site,” “penetration site,” “target site,” and “installation site” may be used in this application to describe a location on a bone at which an intraosseous device may be inserted or drilled into the bone and associated bone marrow. Insertion sites, penetration sites, target sites, and installation sites are generally covered by skin and soft tissue. The term “target area” may be used in this application to describe selected portions of a bone cavity or locations in a bone cavity from which associated bone marrow may be harvested in accordance with teachings of the present disclosure.

A powered driver may be used to insert the IO device incorporating teachings of the present disclosure into a selected target area or target site in the span of a few seconds. However, various teachings of the present disclosure are not limited to use with powered drivers. Manual drivers and mechanically assisted drivers, such as spring powered drivers, may also be used with IO devices incorporating teachings of the present disclosure. Such manual drivers may be used with the IO devices of the present disclosure at times when availability or advisability of having a battery powered driver for interosseous access is not possible. Such conditions may involve military special operations where extreme temperatures and severe weight restrictions limit what can be carried into battle. The same may be true for civilian emergency medical services (EMS) or first responders where long shelf life and infrequent use make the convenience of a battery powered driver impractical. When a manual driver is used, manual force may be exerted on a handle or grip to insert a penetrator or needle into the bone to access the bone marrow. A manual driver may also serve as a useful backup in cases where a battery powered driver fails to function, for example, due to a depleted power supply. Thus, a bone may be penetrated and associated bone marrow accessed using the IO devices of the present disclosure with either driver. For some applications, the IO device may be formed with a first end operable to penetrate bone and/or associated bone marrow, and a second end operable to releasably engage a driver, such as a powered driver or a manual driver.

IO needle sets and other IO devices incorporating teachings of the present disclosure may include a first IO device such as a cannula, catheter, or outer penetrator, and a second IO device such as a stylet, trocar, or inner penetrator. Various types of cutting surfaces may be formed proximate a first end of the first IO device and a first end of the second IO device. The cutting surface of the first IO device and the cutting surface of the second IO device may cooperate with each other to penetrate bone and/or associated bone marrow. A first connector or first hub may be used to releasably engage the first IO needle or IO device with the second IO needle or IO device. For example an IO needle set may include a first connector or a first hub with a generally hollow cannula, catheter or outer penetrator attached thereto and extending from a first end of the first hub. A second end of the first hub may be operable to be releasably engaged with a first end of a second connector or a second hub. A stylet, trocar, or inner penetrator may also be attached to and extend from the first end of the second hub. The second end of the first hub may include an opening sized to allow inserting the stylet, trocar or inner penetrator through the opening and a lumen in the cannula, catheter or outer penetrator. A second end of the second hub may be operable to be releasably engaged with a drive shaft extending from a powered driver or a manual driver.

While various features of the present disclosure may be described with respect to IO devices depicted in the figures, the present disclosure is not limited to such IO devices. A wide variety of IO devices may be formed in accordance with teachings of the present disclosure with various dimensions and/or configurations.

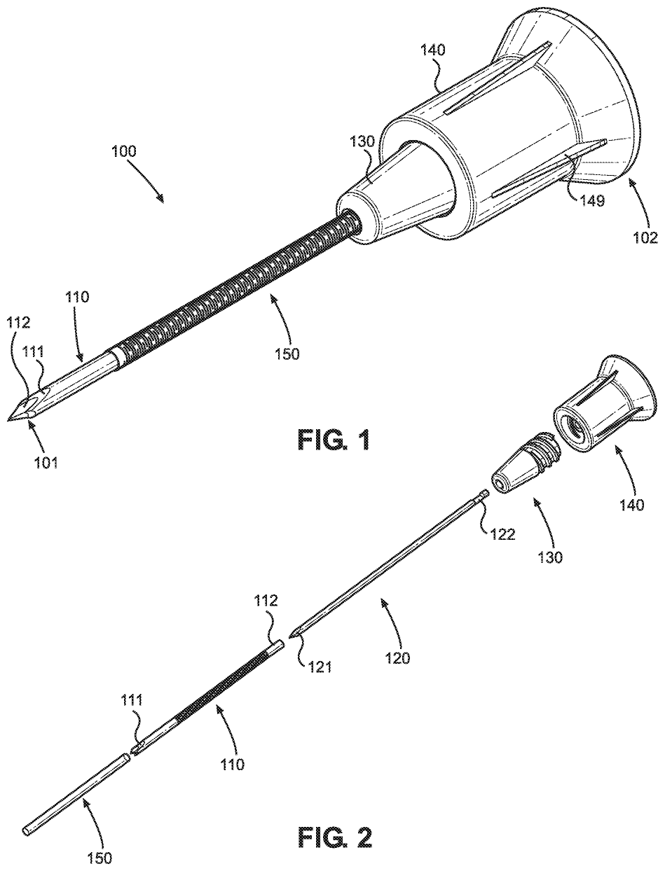

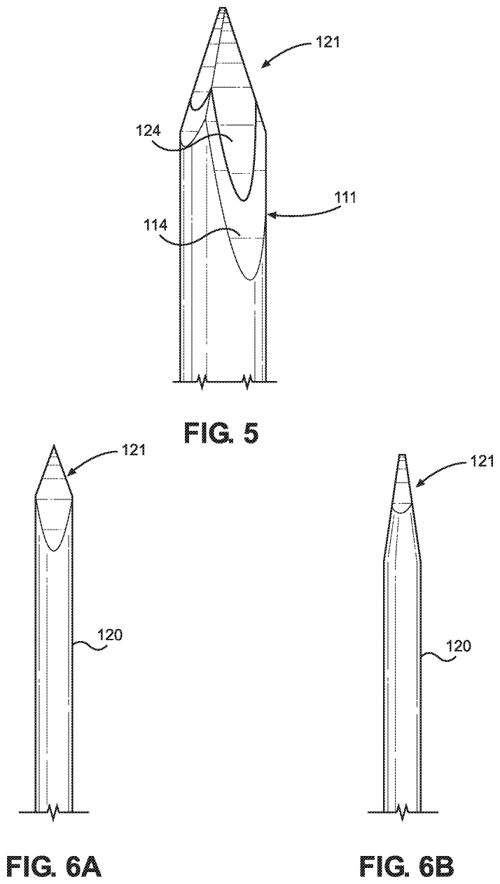

A penetrator assembly, such as IO needle sets 100, 100a, 100b, and 100c, represent only some examples of IO access devices formed in accordance with teachings of the present disclosure. Referring to FIGS. 1 and 2, the penetrator assembly 100 comprises a flexible outer penetrator or cannula 110, and a rigid inner penetrator or stylet 120. A first end 111 of the cannula 110 and a first end 121 of the stylet 120 may be operable to penetrate a bone and associated bone marrow. In particular, the first ends 111, 121 of the cannula and the stylet define respective cutting tips. The stylet 120 includes a rigid longitudinal body and is configured to be slidably and releasably disposed within a longitudinal bore or lumen 118 of the cannula 110. Various features of the first end 111 of the cannula 110 and the first end 121 of the stylet 120 are shown in more detail in FIGS. 5-6, as will be discussed below. In some implementations, fluid ports may be provided at various locations on the cannula body, including at the first end 111 of the cannula. A first end 101, or distal end, of the penetrator assembly 100 may correspond generally with first end 111, or distal end, of the cannula 110 and the first end 121, or distal end, of the stylet 120. A second end 102, or proximal end, of the penetrator assembly may correspond generally with the second end 112, or proximal end, of the cannula 110 and the second end 122, or proximal end, of the stylet 120. The second end 102 of the penetrator assembly may be operable to releasably attach to a driver, as will be discussed in further detail below.

The penetrator assembly 100 further comprises a hub 130 and a connector 140. The second end 112 of the cannula 110 is opposite from the first end 111 of the cannula and may be securely engaged with the a first end 131, or distal end, of the hub 130. In some implementations, the second end 112 of the cannula may be bonded to a portion of the hub within the first end 131 of the hub via UV adhesive or other type of adhesive. A second end 122 of the stylet 120 is opposite from first end 121 of the stylet and may be securely engaged with a first end 141, or distal end, of the connector 140. In some implementations, the second end 122 of the stylet may be bonded to a portion 145 of the connector disposed within the first end 141 of the connector via UV adhesive or other type of adhesive.

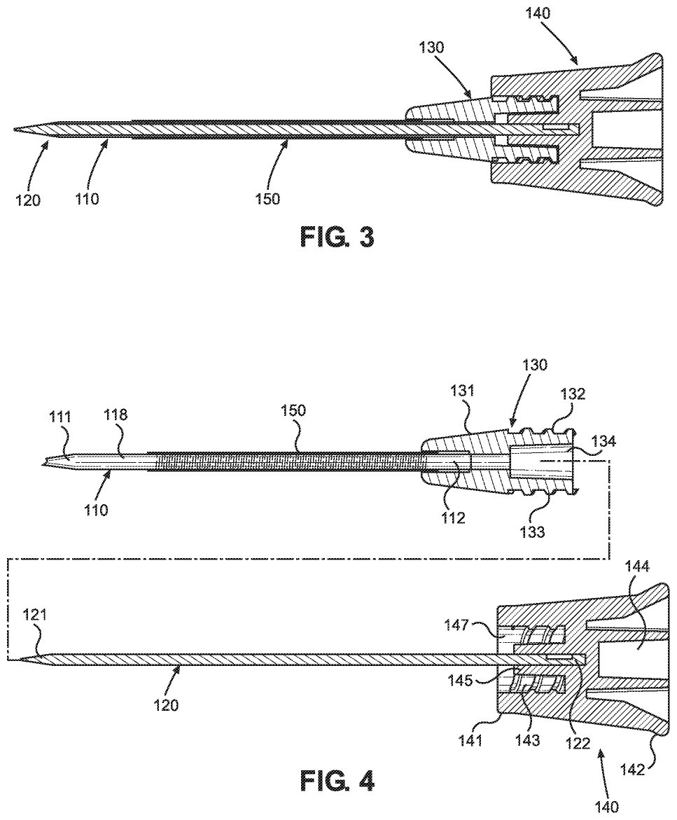

As shown in FIGS. 3 and 4, the cannula 110 may extend longitudinally from the first end 131 of the hub 130, and the stylet 120 may extend from the first end 141 of the connector 140. The second end 132, or proximal end, of the hub 130 may include a Luer lock fitting which may be releasably engaged with a corresponding Luer lock fitting disposed within the first end 141 of the connector 140. More particularly, the second end 132 of the hub 130 may include a male threaded portion 133, and the first end 141 of the connector 140 may include a female threaded portion 143, such that male threaded portion 133 of the hub 130 is operable to removably connect to the female threaded portion 143 of the connector 140 resulting in a threaded connection between the second end 132 of the hub 130 and the first end 141 of the connector 140. The Luer lock fitting 133 disposed on the second end 132 of the hub 130 may be operable to be releasably engaged with a standard syringe type fitting and/or a standard intravenous (IV) connection and associated fluid tubing for aspirating the IO space through the cannula lumen 118, delivering medication to the IO space through the cannula lumen 118, or capturing a biopsy specimen of a bone and associated bone marrow. When the hub 130 is attached to the connector 140, the stylet 120 is disposed within the cannula 110 such that the stylet extends longitudinally from the first end 141 of the connector 140 and the first end 131 of the hub 130.

Various types of receptacles may be disposed in the second end 142, or proximal end, of the connector 140 for use in releasably engaging the connector with a drive shaft of a manual driver or a powered driver. For example, a driver, such as a manual or powered driver, having a drive shaft with a tapered portion may be operable to be releasably engaged with a receptacle 144 having a corresponding generally tapered configuration disposed in the second end 142 of the connector 140. In some implementations, a driver may be secured to an intraosseous device by a magnet disposed on the end of the tapered shaft extending from the driver and a corresponding metal or magnetic disk disposed within the receptacle 144 in the intraosseous device.

As previously discussed with reference to FIGS. 3 and 4, the penetrator assembly 100 may include the outer penetrator 110 such as a cannula, hollow tube or hollow drill bit, and the associated hub 130. The penetrator assembly 100 may also include the inner penetrator 120, such as a stylet or trocar, and the associated connector 140. Various types of stylets and/or trocars may be disposed within the outer penetrator. For some applications, outer penetrator or cannula 110 may be described as a generally elongated tube sized to receive the inner penetrator or stylet 120 therein. Portions of inner penetrator 120 may be disposed within the longitudinal passageway 118 extending through outer penetrator 110. The outside diameter of the inner penetrator 120 and the inside diameter of the longitudinal passageway 118 may be selected such that the inner penetrator 120 may be slidably disposed within the outer penetrator 110.

A metal disc may be disposed within the opening 144 in the second end 142 of the connector 140 for use in releasably attaching the connector 140 with a magnetic portion of a drive shaft of a manual driver or a powered driver. In some implementations, the drive shaft of the driver may be magnetized. The second end 122 of the inner penetrator 120 is preferably spaced from the metal disc with insulating or electrically nonconductive material disposed therebetween.

FIG. 5 shows an example of the cutting surfaces and tips which may be formed at the first ends 111, 121 of the respective outer 110 and inner 120 penetrators in accordance with teachings of the present disclosure. The first end 111 of the outer penetrator 110 and/or the first end 121 of the inner penetrator 120 may be operable to penetrate bone and associated bone marrow. The configuration of tips and/or cutting surfaces at the first end 111, 121 of the respective outer 110 and inner 120 penetrators may be selected to penetrate a bone or other body cavities with minimal trauma.

The respective first ends 111, 121 of the outer penetrator 110 and the inner penetrator 120 may be ground together as one unit during an associated manufacturing process such that when the hub 130 is securely fastened to the connector 140, the cannula or outer penetrator 110 and the stylet or inner penetrator 120 are disposed relative to each such that the respective cutting surfaces of the outer and inner penetrators are substantially coplanar. Accordingly, the stylet 120 has an angled/beveled tip that matches an angled/beveled tip of the cannula 110.

In other words, the first end 111 of the cannula 110 and the first end 121 of the stylet 120 and may be ground at the same time to form adjacent cutting surfaces 114, 124. Grinding the respective ends 111, 121 at the same time may result in forming a single cutting unit to form generally matching cutting edges as shown in FIG. 5. In other implementations, the respective first ends 111, 121 of the outer penetrator 110 and the inner penetrator 120 may be ground separately during an associated manufacturing process such that when the hub 130 is securely fastened to the connector 140, the cannula or outer penetrator 110 and the stylet or inner penetrator 120 are disposed relative to each such that the respective cutting surfaces 114, 124 of the outer and inner penetrators are substantially coplanar. Accordingly, the stylet 120 has an angled/beveled tip that matches an angled/beveled tip of the cannula 110. Further, a cross-section of the first end 111 of the outer penetrator 110 may be trapezoid-shaped and may include one or more cutting surfaces. Similarly, a cross-section of the first end 121 of the inner penetrator 120 may be trapezoid-shaped and may include one or more cutting surfaces.

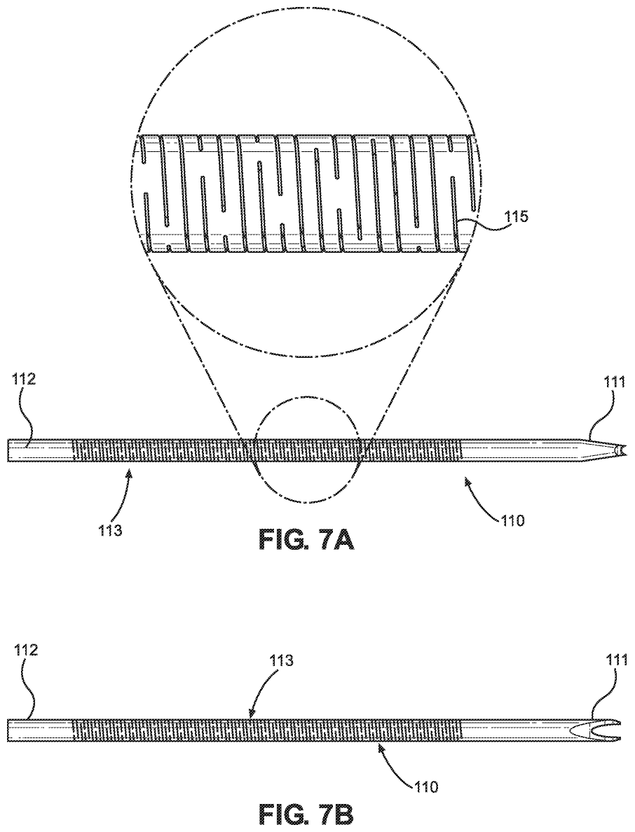

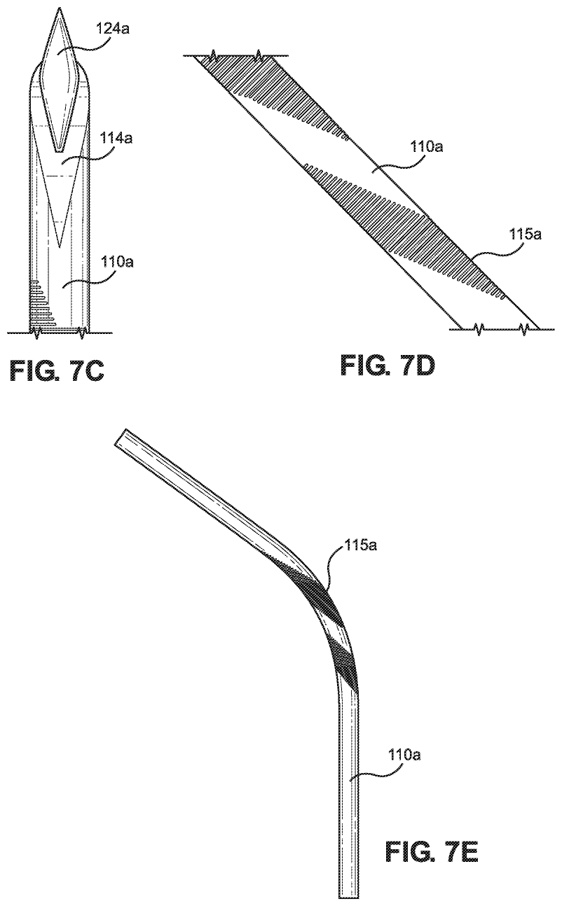

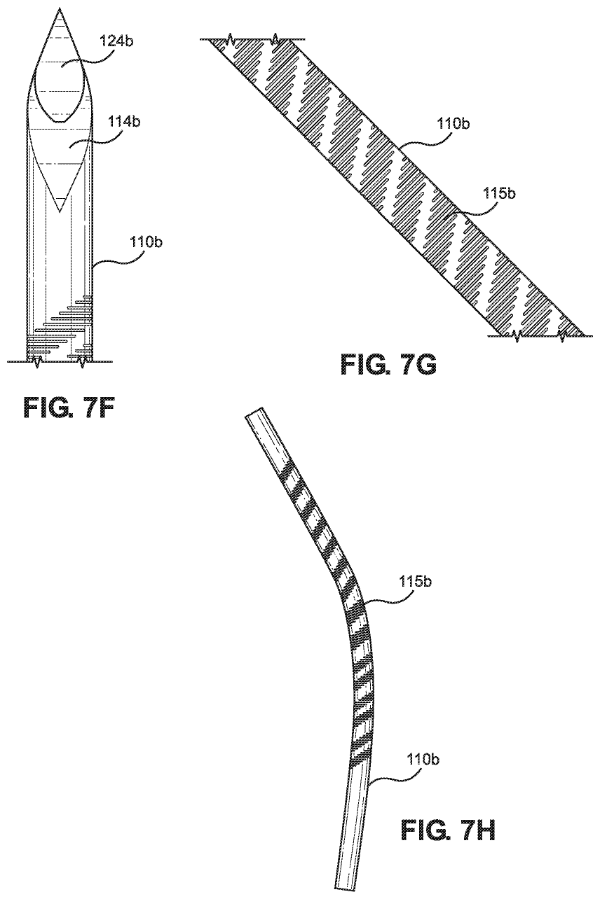

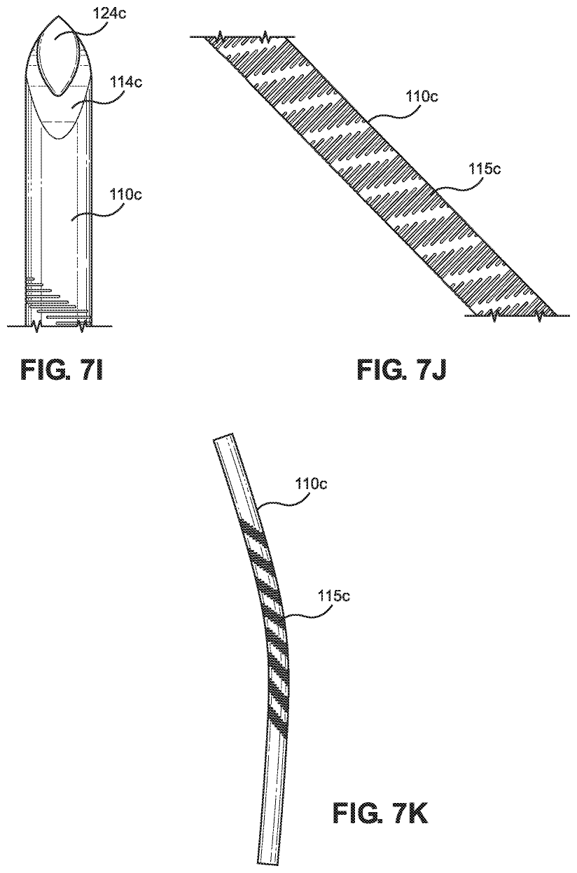

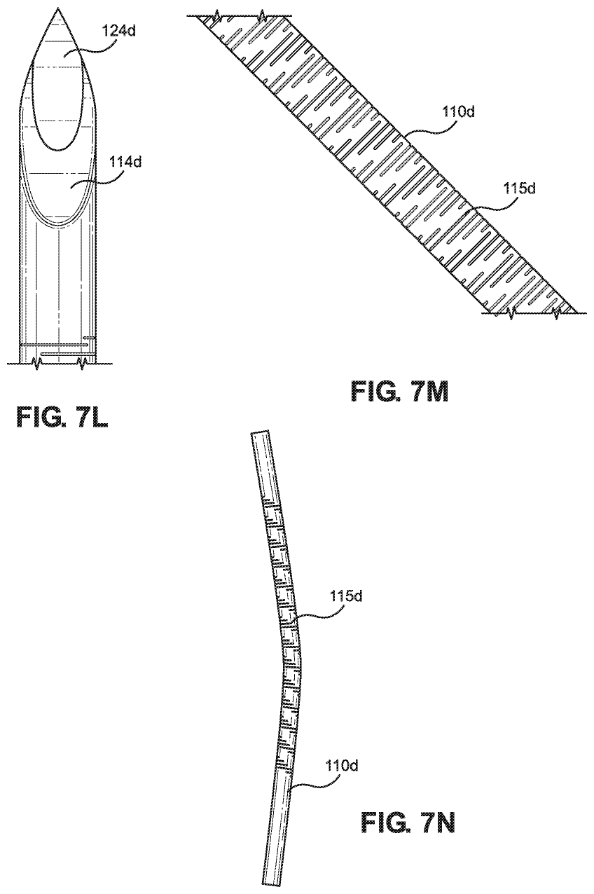

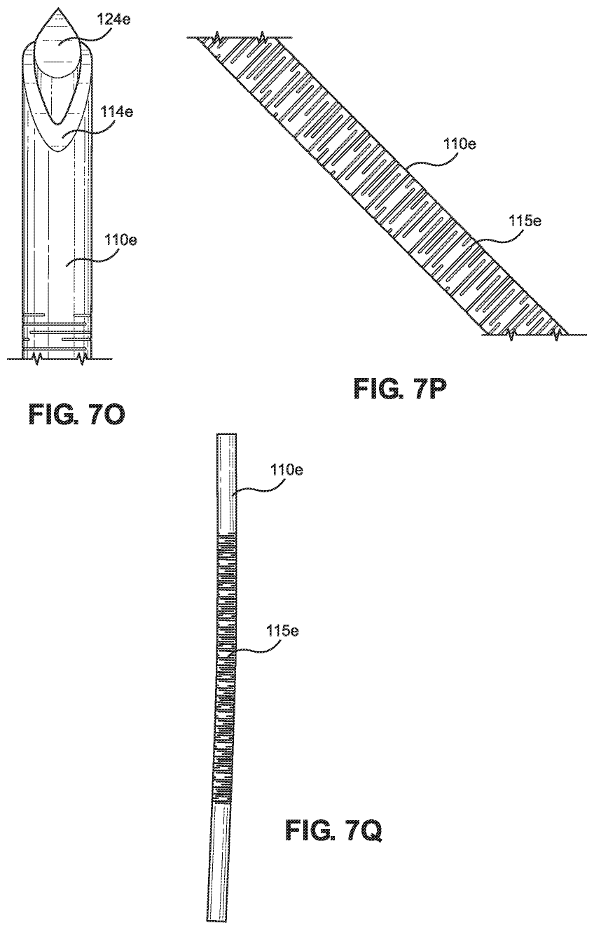

As shown in FIGS. 6A and 6B, for instance, the cutting surfaces on the first end 121 of the inner penetrator 120 include a first face and an adjacent second face, where the first face is longer than the second face. Similarly, the cutting surfaces on the first end 111 of the outer penetrator 110 include a first face and an adjacent second face, where the first face is longer than the second face. Providing a matching fit allows respective tips at the first ends 111, 121 of the outer and inner penetrators to act as a single drilling unit which facilitates insertion and minimizes damage as portions of penetrator assembly 100 are inserted into a bone and associated bone marrow. The inner and outer penetrators may be formed from stainless steel, titanium, or other biocompatible materials of suitable strength and durability to penetrate bone. As shown in FIGS. 7C, 7F, 7I, 7L, and 7O, respective cutting surfaces of the outer and inner penetrators may be sized and shaped in various configurations in order to achieve a desired effectiveness of bone penetration as well as to maintain strength of the needle tip during insertion into, and withdrawal out of, the bone and associated bone marrow.

The second end 132 of the hub 130 may be operable for releasable engagement or attachment with the associated connector 140. The first end 131 of the hub 130 may have a size and configuration compatible with an associated insertion site for the outer penetrator 110. The connector 140 may include an enlarged tapered portion adjacent to the second end 142. A plurality of longitudinal ridges 149 may be formed on an exterior surface of the connector 140 to allow an operator to grasp the associated penetrator assembly 100 during attachment with a drive shaft of a driver. The longitudinal ridges 149 also allow the connector 140 to be grasped for disengagement from the hub 130 when the outer penetrator 110 has been inserted into a bone and associated bone marrow so that the outer penetrator can remain indwelling at the insertion site.

The first end 141 of the connector 140 may include an opening 147 sized to receive the second end 132 of the hub 130 therein. Threads 143 may be formed in the opening 147 adjacent to the first end 141 of the connector 140. The threaded fitting 143 may be used to releasably attach the connector 140 with the corresponding threaded fitting 133 adjacent to the second end 132 of the hub 130. The second end 132 of the hub 130 may include a threaded portion 133 or other suitable fitting formed on the exterior thereof. Further, the second end 132 of the hub may have a generally cylindrical opening 134 configured to matingly receive a corresponding generally cylindrical protrusion 145 disposed within the first end 141 of the connector 140, i.e., defining a pin type configuration. The protrusion 145 may include a receptacle configured to securely receive the second end 122 of the stylet 120, which may be bonded to the connector via UV adhesive or other type of adhesive.

In some implementations, the first end 131 of the hub 130 may include a flange. Additionally, an angular slot or groove configured to receive one end of protective cover or needle cap may be formed in the first end 131 of the hub 130. In some implementations, an angular slot or groove configured to receive one end of protective cover or needle cap may be formed in the first end 141 of the connector 140. Such a slot or groove may be used to releasably engage a protective cover with the penetrator assembly 100. In some aspects, the cover may be a generally hollow tube having a rounded end. The cover may be disposed within an associated slot to protect portions of the outer penetrator 110 and the inner penetrator 120 prior to attachment with an associated driver. The cover may include a plurality of longitudinal ridges formed on the exterior thereof. The longitudinal ridges cooperate with each other to allow installing and removing the cover or needle cap without contaminating portions of an associated penetrator. The cover may be formed from various plastics and/or metals.

The dimensions and configuration of the first end 131 of the hub 130 may be varied to accommodate various insertion sites and/or patients. For instance, the first end 131 of the hub 130 may further include an annular flange or other configuration compatible for contacting a patient's skin. In some implementations, the first end 131 does not include an annular flange or other configuration compatible for contacting a patient's skin. A passageway through the hub 130 extends from the first end 131 through the second end 132. The dimensions and configuration of the inside diameter of the passageway may be selected to securely engage the outside diameter of the second end 112 of the cannula or outer penetrator 110. In some aspects, the second end 112 of the cannula or outer penetrator 110 may be bonded to the hub via UV adhesive or other type of adhesive.

For some applications, the threaded connection or fittings 133 of the first end 132 of the hub 130 may allow for attachment with various types of Luer locks and/or Luer fittings associated with intravenous tubing or a syringe. For instance, once the outer penetrator or cannula 110 is inserted into bone and associated bone marrow, various types of connections may be used to communicate fluids to the bone marrow via the outer penetrator or cannula by connecting an intravenous tubing to the outer penetrator. A right angle connector may also be used to connect intravenous tubing to the outer penetrator at an angle that will not kink or pinch off the lumen of tubing. In some aspects, a lock nut may be used to engage such a right angle connector with the hub 130. Various other types of connectors may be used to communicate fluids between the outer penetrator 110 and the intravenous tubing so that the tubing may be used to provide intravenous fluids and/or medications to the associated bone marrow. Tubing may also be used in withdrawing a sample of blood or bone marrow from the IO space. Other connectors or adapters may also be used to connect a penetrator to an intravenous tubing, other types of tubing, and/or a syringe.

The outer penetrator or cannula 110 comprises an intermediate portion 113 provided between the first end 111 and the second end 112, as shown in FIGS. 7A and 7B. The cannula 110 may be formed from stainless steel or other suitable biocompatible material. While the first and second ends 111, 112 of the cannula are rigid and non-flexible, the intermediate portion 113 of the cannula is configured to deform by bending or flexing. In particular, the intermediate portion 113 comprises a plurality of cuts or slits 115 disposed on exterior portions thereof that allow for the cannula 110 to bend. In some implementations, the cuts are through-cuts as shown in FIG. 7A. In some implementations, at least some of the cuts may be notches or other thinning portions of the cannula material. Thus, the cuts may penetrate the cannula wall completely, or they may be perforations or grooves etched in the cannula wall, or a combination of both. Incision of the cuts may be formed, for example, by lasers, chemical etching, or water blasting, among other techniques. The cuts or slits 115 allow the intermediate portion 113 of the cannula to act as a plastically deformed segment where the overall deformation of the cannula occurs. The configuration of the cuts or slits, including their size, shape, and pattern, are designed to facilitate a particular direction and/or degree of deformation of the cannula.

Joints formed by the cuts or slits 115 in the cannula support deformation outside a patient's body or adjacent to a point of entry of the cannula into the body. Accordingly, the cannula 110 is able to remain inserted into a target area in-situ in the patient for longer periods of time since the cannula can bend or flex to accommodate movements made by the patient. For instance, a standard rigid cannula inserted into a humerus of a patient may become dislodged upon movement of the patient's arm. However, the flexible cannula 110 of the present disclosure would be prevented from being dislodged from the patient's humerus because it would be able to bend or flex to accommodate movement of the patient's arm, such as when the arm is raised above the patient's head.

The pattern, size, and shape of the laser cuts or slits 115 may be selected in order to provide a desired degree of flexibility while also taking into account expected applied forces. Further, the pattern and/or design of the cuts or slits 115 may be selected to provide a particular insertion and withdrawal integrity of the cannula to control how much the shape of a bent or flexed cannula is able to recover, i.e., whether the shape of the cannula can recover partially or fully.

For example, an outer penetrator 110a comprising the laser cut design and/or pattern 115a illustrated in FIG. 7D is configured to have strong insertion and withdrawal integrity, and minimal or below average shape recovery. In other words, once the cannula 110a is bent it will generally stay in the bent shape, as shown in FIG. 7E. In another implementation, an outer penetrator 110b comprising the laser cut design and/or pattern 115b illustrated in FIG. 7G is configured to have medium insertion and withdrawal integrity, and approximately average shape recovery as shown in FIG. 7H. In another implementation, an outer penetrator 110c comprising the laser cut design and/or pattern 115c illustrated in FIG. 7J is configured to have medium insertion and withdrawal integrity, and average shape recovery as shown in FIG. 7K. In another implementation, an outer penetrator 110d comprising the laser cut design and/or pattern 115d illustrated in FIG. 7M is configured to have medium insertion and withdrawal integrity, and approximately average shape recovery as shown in FIG. 7N. In another implementation, an outer penetrator 110e comprising the laser cut design and/or pattern 115e illustrated in FIG. 7P, the laser cut design is configured to have medium insertion and withdrawal integrity, and maximal or above average shape recovery as shown in FIG. 7Q.

Each laser cut design and/or pattern of the slits shown in the figures allows the cannula to be left in an insertion site as initially inserted, such that the cannula extends away from the insertion site, and thus the associated hub is correspondingly spaced away from the patient. Each laser cut design and/or pattern of the slits shown in the figures also allows the cannula to be bent down and fixed in a desired orientation, i.e., against the patient such that the cannula and the associated hub remain close to the patient, and therefore are less obtrusive and thus less likely to be dislodged from the insertion site by accidentally bumping or knocking them.

Referring generally back to FIG. 7A. the deformable cannula 110 is further configured to withstand the loads acting on it during usage, for example torque during insertion into a bone and/or removal therefrom. In other words, the particular design and pattern of laser cuts or slits on the cannula is optimized in order to withstand varying levels of torque applied to the cannula during an insertion procedure. Additionally, each joint formed by the slits 115 permits a deformation of the cannula. The length of the cannula subject to deformation may vary with the total deformation implemented and/or the number of slits 115 provided in the intermediate section 113. In one example, the pattern of laser cut slits 115 may have a cut/uncut total of 120 degrees, which allows for three cut lines on the one pitch, and incrementing rotationally by 90 degrees. Such a laser cut pattern allows the cannula to withstand greater torque due to an extra spine on every second pitch.

Turning again to FIGS. 1-4, the rigid stylet 120 is configured to support the cannula 110 during insertion and further prevents deformation of the cannula until such deformation is desired, i.e., by removing the stylet from the cannula before deformation is undertaken. During an insertion procedure, the hub 130 of the cannula 110 is engaged to the connector 140 such that the rigid stylet 120 of the connector is received within the longitudinal bore 118 of the cannula to provide sufficient rigidity to the intermediate portion 113 of the cannula in order for the cannula to withstand the force or torque applied thereto by the manual or powered driver. Accordingly, the stylet 120 is configured to add stiffness to the intermediate portion 113 of the cannula 110 during an insertion procedure so that the cannula does not inadvertently bend out of shape due to the force and/or torque applied via a manual or powered driver. In other words, the rigid stylet provides stability and rigidity to counteract the force and/or torque applied to the flexible cannula as the penetrators are pushed and/or rotated (i.e., in a clockwise direction) by the driver to penetrate into dense bone.

Once the tips of the penetrators are inserted into the IO space, the stylet 120 is removed so that the cannula 110 can remain in the same inserted position or be bent over against the patient's skin to accommodate an intravenous tubing or other IO device, while also creating a lower profile of the cannula against the patient's skin so that the cannula and associated hub are less obtrusive. The bent portion of the cannula may be taped down against the patient's skin to further secure it in place. Once the cannula is inserted in-situ in the target site of the patient, fluid leakage may occur through the slits 115, for example, when aspirating blood or bone marrow, or when administering medication. At least some of the cuts tend to at least partially close during bending of the cannula depending on the degree of bending that the cut is subjected to. For instance, slits on the inner side of the bend resulting from flexing the cannula tend to close as a result of the deformation. Such partial closing reduces leaking of material passing through the cannula. However, certain slits may open to a larger degree when the cannula is flexed, such as slits on the outer side of the bend, and thus the fluid leakage through the opened slits may correspondingly increase. For instance, slits on the outer side of the bend caused by flexing the cannula to remain the same size or open slightly as a result of the deformation.

A flexible sleeve 150 may be provided over an exterior surface of the intermediate portion 113 of the cannula to prevent or minimize fluid leakage, or in other words the flexible sleeve may be provided over the cuts or slits 115. Thus, both the intermediate section 113 of the cannula and the deformable sleeve 150 are adapted to bend, and may remain inside and/or outside of an insertion site on the patient's body. Accordingly, the sleeve 150 is configured to cover the cuts 115 to reduce leaking of material injected or withdrawn through the cannula 110. The covering sleeve 150 may be transparent. The covering sleeve 150 may be a heat-shrink tubing comprising a polymer, such as fluorinated ethylene propylene. Heat shrinking the sleeve 150 over the exterior of the intermediate portion 113 of the cannula 110 ensures a fitted fluid seal is formed over the laser cut zone of the cannula so that fluid does not leak through the associated slits 115. Heat shrinking the sleeve over the slits in the cannula prior to use also ensures that the sleeve adheres to the cannula with sufficient strength to prevent or reduce leakage of material passing through the cannula at a high pressure. The sleeve may be heat shrunk during manufacturing (e.g., with a heat gun) so that it is sufficiently adhered to the cannula during an insertion procedure.

In some implementations, the sleeve 150 may extend beyond the intermediate portion 113 of the cannula having the slits 115. In some implementations, the sleeve 150 may be non-compliant so that its diameter remains the same when a material flows through the cannula at a high pressure. Further, the sleeve 150 may comprise a sufficient wall thickness to ensure stability under the a high pressure flow of material through the cannula 110.

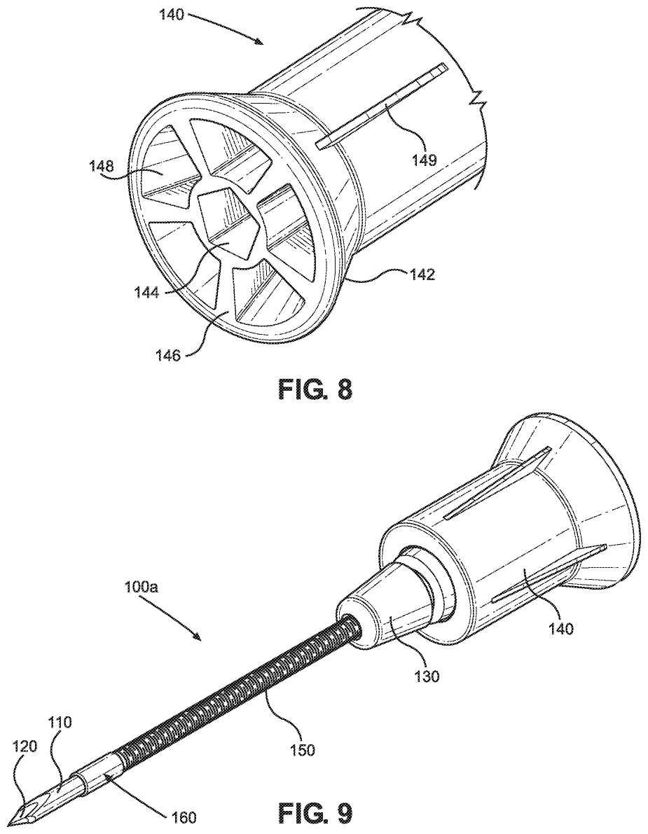

Referring to FIG. 8, the second end 142 of the connector 140 is shown. As previously described, a drive shaft or attachment may be releasably engaged with the second end 142 of the connector 140, and the inner penetrator or stylet 120 extends from the first end 141 of the connector. The connector 140 and the hub 130 may be releasably engaged with each other by Luer type fittings, threaded connections, or other suitable fittings formed on the first end 141 of the connector and the second end 132 of the hub, respectively.

An opening or recess 144 may be formed in the second end 142 of the connector to receive an associated drive shaft of a manual or powered driver. The opening 144 may be formed with various configurations and/or dimensions. In some implementations, the opening 144 may include a passageway or channel sized to receive portions of the drive shaft. One or more webs 146 may be formed in the second end 142 of the connector and extend radially away from the opening 144. Open segments or void spaces 148 may be formed between the webs. Respective projections of a drive shaft may be releasably engaged with the webs 146 and void spaces 148. The opening 144 and associated webs 146 may be used to releasably couple the connector 140 with either a manual driver or a powered driver.

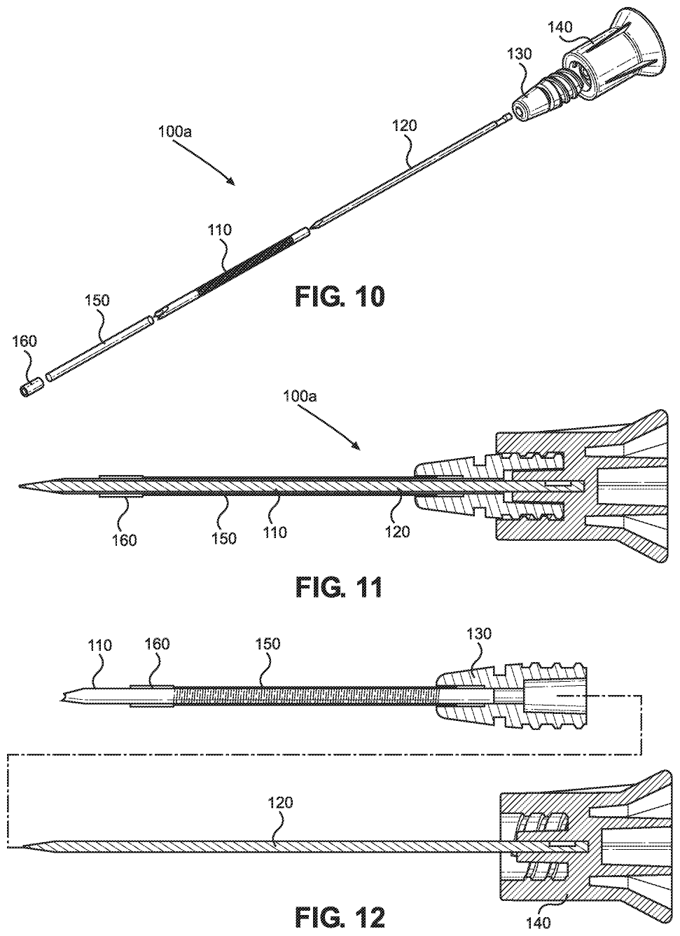

Turning to FIGS. 9-12, another implementation of a penetrator assembly, such as IO needle set 100a, is illustrated. The IO needle set 100a is substantially similar to the IO needle set 100 described in detail above, and further includes a depth control collar 160 fixed to the first end 111 of the outer penetrator or cannula 110. The depth control collar 160 is configured to limit the depth of insertion of the inner and outer penetrators during an insertion procedure. The collar 160 may have a generally elongated, hollow configuration compatible with engaging the outside diameter of outer penetrator 110. The collar 160 may be welded to the first end 111 of the outer penetrator 110 to form an integral unit therewith. A distal end of the collar is spaced from the tip portion of the inner and outer penetrators at a predetermined distance corresponding to the depth of insertion for the first end of the inner and outer penetrators into the IO space. Accordingly, the depth of penetration into a bone and associated bone marrow may be determined by the distance between the distal edge of the collar 160 and the distal or extreme end of the needle tip formed at the first ends 111, 121 of the cannula and stylet.

During an insertion procedure, the distal end of the collar 160 is configured to contact the bone, thus preventing further penetration of the inner and outer penetrators from being inserted any further into the IO space. Accordingly, the collar 160 is configured to limit a depth of insertion of the inner and outer penetrators into bone and associated bone marrow. The collar 160 may be formed from various materials including stainless steel, titanium, or other materials, such as those used to form the outer penetrator 110. Since the collar 160 will generally be securely engaged with the exterior surface of the outer penetrator 110, the outer penetrator 110 and the collar 160 will rotate concurrently with each other in response to rotation of a driver. For instance, when using a manual driver, a user may insert the needle set 100a through tissue and bone until the distal edge of the collar 160 contacts the bone, thus preventing further insertion of the needle set.

Further, providing the collar 160 on exterior portions of the outer penetrator 110 allows the penetrators 110, 120 to be satisfactorily used to access bone marrow in an IO space by selectively limiting the depth of penetration. The sleeve 150 covering the laser cut slits in the cannula 110 may have an outer diameter that is less than or equal to the size of an outer diameter of the collar 160 in order to protect the sleeve from damage during an insertion procedure.

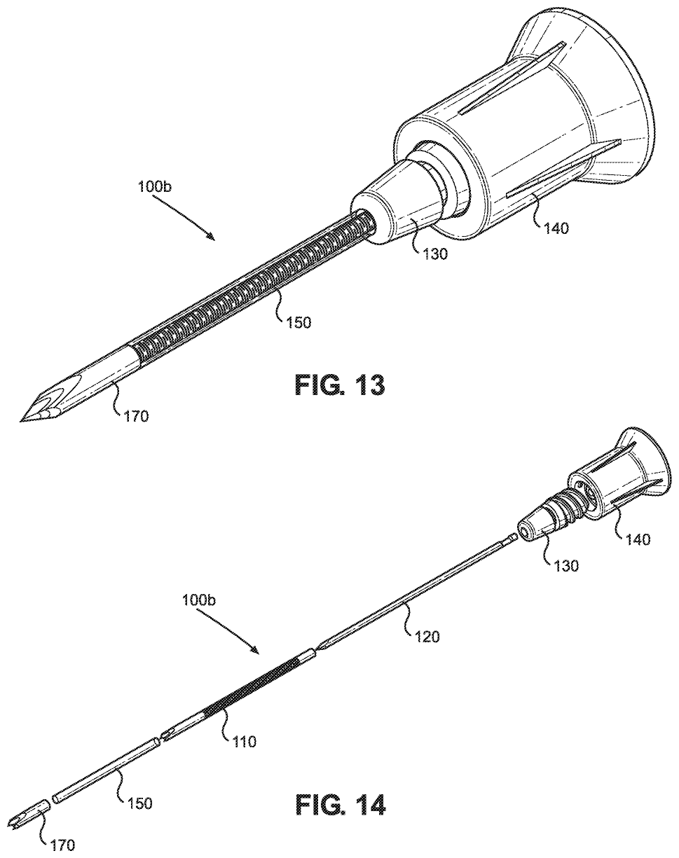

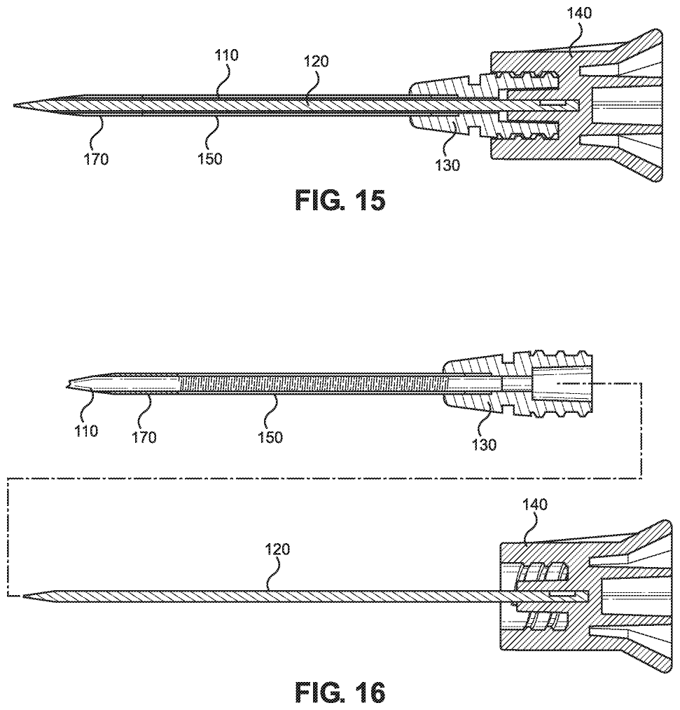

Another implementation of a penetrator assembly, such as IO needle set 100b, is illustrated in FIGS. 13-18. The IO needle set 100b is substantially similar to the IO needle set 100 described in detail above, and further includes a distal cutting sleeve 170 fixed to the first end 111 of the outer penetrator or cannula 110. The distal cutting sleeve 170 may be welded to the first end 111 of the outer penetrator to form an integral unit therewith.

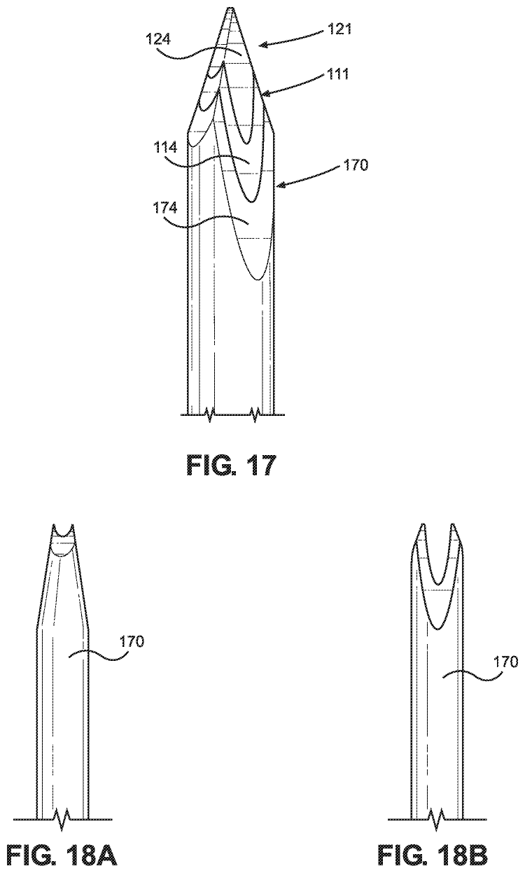

FIG. 17 shows an example of the cutting surfaces and tips which may be formed at the first ends 111, 121 of the respective outer 110 and inner 120 penetrators and the distal cutting sleeve 170. The cutting surfaces 114, 124 of the outer and inner penetrators 110, 120 and the cutting surface 174 of the distal cutting sleeve may be ground together as one unit during an associated manufacturing process such that when the hub 130 is securely fastened to the connector 140, the cannula 110 and the stylet 120 and the distal cutting sleeve 170 are disposed relative to each such that their respective cutting surfaces are coplanar. In other words, the angled/beveled cutting surfaces at the tip ends of the inner penetrator, the outer penetrator, and the distal cutting sleeve are configured to match each other.

In other implementations, the respective first ends 111, 121 of the outer penetrator 110 and the inner penetrator 120, as well as the distal cutting sleeve 170, may be ground separately during an associated manufacturing process such that when the hub 130 is securely fastened to the connector 140, the respective cutting surfaces 114, 124, 174 of the outer penetrator 110, the inner penetrator 120, and the distal cutting sleeve 170 are substantially coplanar. Accordingly, the stylet 120 has an angled/beveled tip that matches an angled/beveled tip of the cannula 110, and which also matches an angled/beveled tip of the distal cutting sleeve 170. Further, a cross-section of the first end 111 of the outer penetrator 110 may be trapezoid-shaped and may include one or more cutting surfaces. Similarly, a cross-section of the first end 121 of the inner penetrator 120 may be trapezoid-shaped and may include one or more cutting surfaces. Likewise, a cross-section of a first end of the distal cutting sleeve 170 may also be trapezoid-shaped and may include one or more cutting surfaces.

Providing a matching fit for each cutting surface of the penetrators 110, 120 and the distal cutting sleeve 170 allows the respective tip portions to act as a single drilling unit which facilitates insertion into a bone and associated bone marrow and therefore also minimizes injury to the patient. Similar to the outer and inner penetrators 110, 120, the distal cutting sleeve 170 may be formed from stainless steel, titanium, or other biocompatible materials of suitable strength and durability to penetrate bone. Further, the flexible sleeve 150 covering the laser cut slits 115 in the cannula may have an outer diameter that is less than or equal to the size of an outer diameter of the distal cutting sleeve 170 in order to protect the sleeve from damage during an insertion procedure.

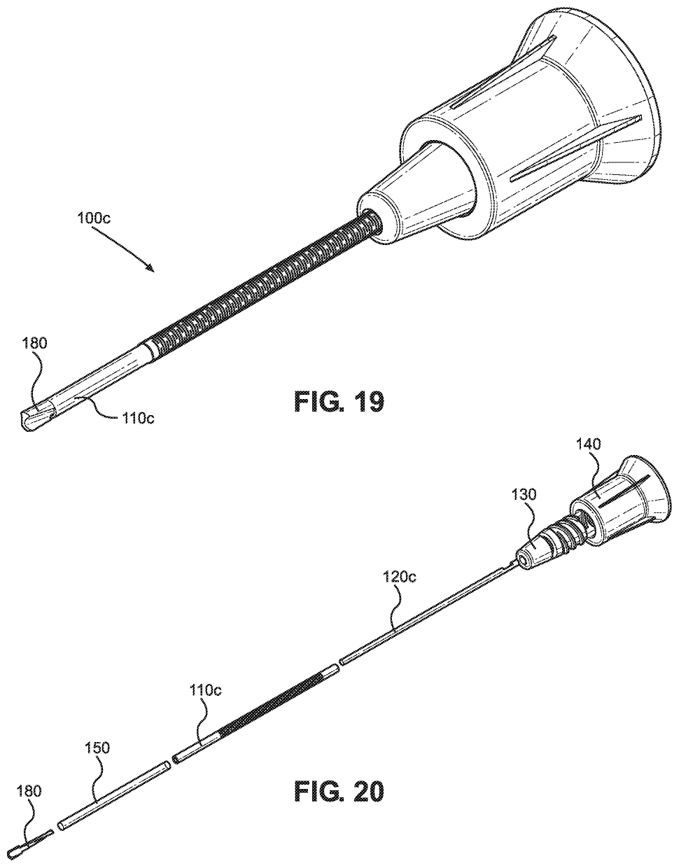

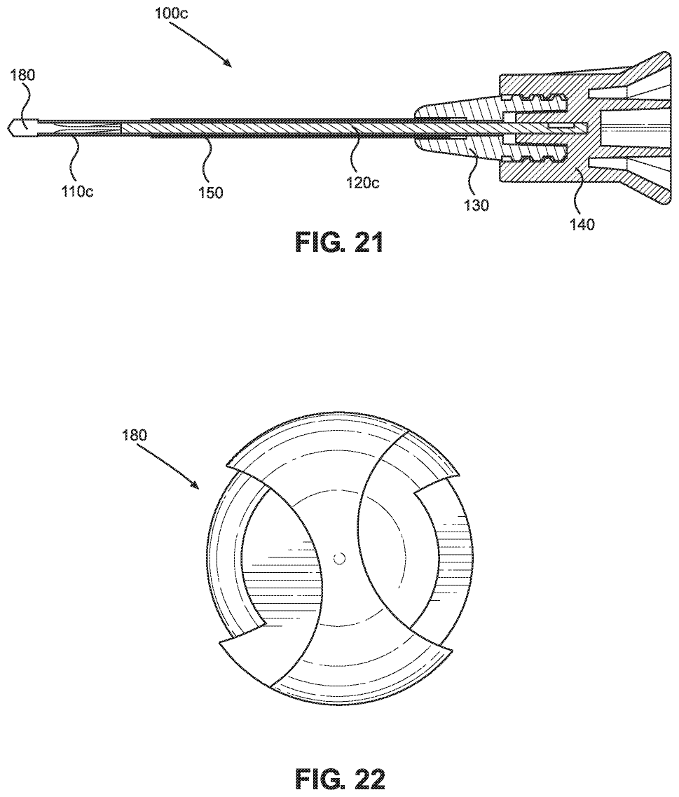

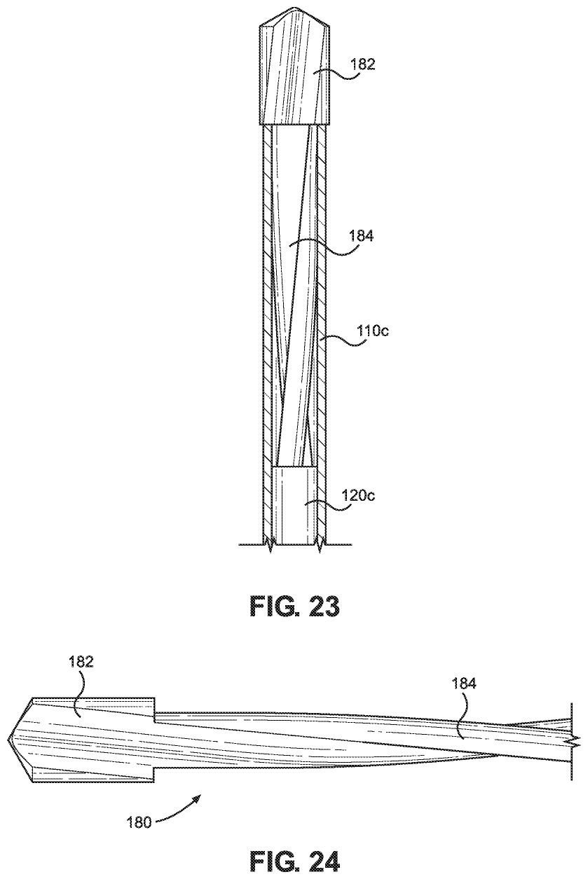

Another implementation of a penetrator assembly, such as IO needle set 100c, is illustrated in FIGS. 19-24. The IO needle set 100c is substantially similar to the IO needle set 100 described in detail above, but includes the following noted differences. In particular, the IO needle set 100c comprises a fluted drill tip 180 fixedly secured to a first end of a flexible cannula 110c. The fluted drill tip 180 may be welded to a first end of the flexible cannula 110c to form an integral unit therewith. Similar to the inner and outer penetrators, the fluted drill tip 180 may be formed from stainless steel, titanium, or other biocompatible materials of suitable strength and durability to penetrate bone. The rigid stylet 120c is used during an insertion procedure to provide structural rigidity to support the flexible cannula 110c and prevent it from bending or deforming. Upon insertion of the needle set 100c into the IO space, the stylet 120c is removed from the cannula 110c to allow the cannula to bend and flex to a desired orientation. Such bending and flexing of the cannula 110c is due to the cuts or slits 115 formed in an intermediate section thereof, as previously described in detail above. In some implementations, the first end, or tip, of the stylet is blunt so that there is no sharp distal tip. Such a blunt tip provides increased safety to the user when the stylet is withdrawn from the cannula.

The fluted cutting tip 180 includes an integrally formed head portion 182 and a longitudinal body portion 184. The cutting flutes continuously extend through the head and body portions. A proximal end of the head portion is fixedly secured to a distal end of the cannula 110c such that the longitudinal body portion 184 is received within the first end of the cannula. Once the needle set 100c is inserted into the IO space and the stylet 120c is withdrawn from the cannula 110c, the fluted cutting tip 180 remains attached to the first end of the cannula such that the fluted cutting tip remains anchored within the IO space.

The cutting flutes define respective channels along both the head and body portions 182, 184 of the fluted tip 180 that allow for blood and/or bone marrow samples to be aspirated from the IO space or for medication to be delivered to the IO space (i.e., material is able to travel along the channels defined by the fluted head and body portions and the interior surface of the cannula). Further, the flexible sleeve 150 covering the laser cut slits 115 in the cannula 110c may have an outer diameter that is less than or equal to the size of an outer diameter of the head portion of the fluted cutting tip 180 in order to protect the sleeve from damage during an insertion procedure.

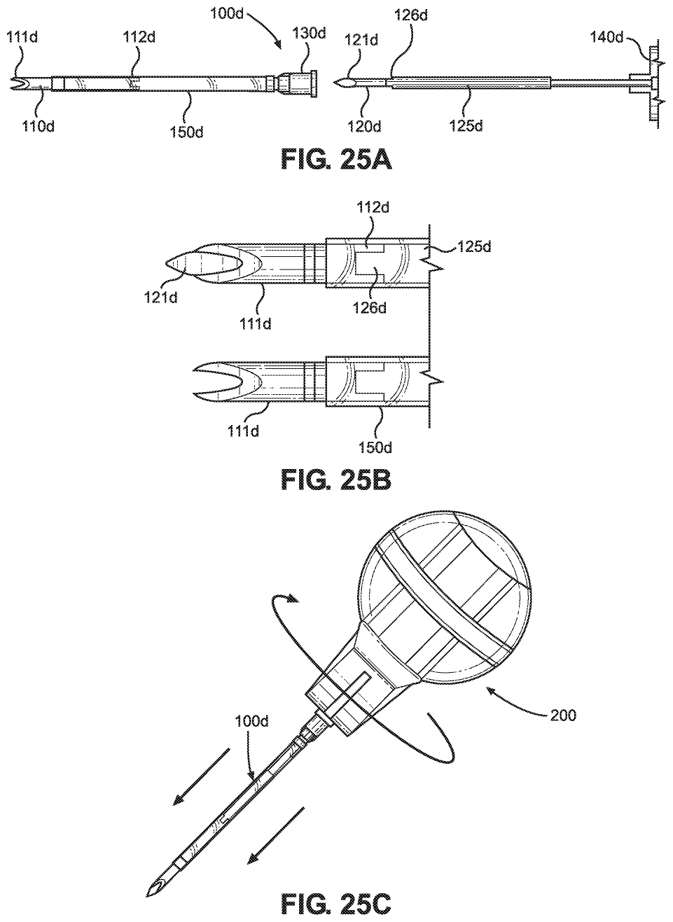

FIG. 25A illustrates another implementation of a penetrator assembly, such as IO needle set 100d. The penetrator assembly 100d comprises an outer penetrator or cannula 110d, and a rigid inner penetrator or stylet 120d. A first end 111d of the cannula 110d and a first end 121d of the stylet 120d may be operable to penetrate a bone and associated bone marrow. In particular, the first ends 111d, 121d of the cannula and the stylet define respective cutting tips, and the stylet 120d is configured to be slidably and releasably disposed within a longitudinal bore or lumen of the cannula 110d, as similarly described above with respect to IO needle sets 100, 100a, 100b, and 100c.

The penetrator assembly 100d further comprises a hub 130d and a connector 140d which may include respective features of the hub 130 and the connector 140 previously described above. A portion of a flexible sleeve or tube 150d may be adhered or bonded to an exterior surface of a portion of the cannula 110d for providing fluid communication between the cannula and the hub 130d. The flexible sleeve 150d is adapted to bend, and may remain inside and/or outside of an insertion site on the patient's body. The sleeve or tube 150 may comprise a transparent polymer to allow for visualization of material, such as bone marrow or medicine, that passes therethrough.

A second end 112d of the cannula 110d is located opposite from the first end 111d of the cannula and defines a female lock portion. The stylet 120d includes a key section 125d having a first end 126d defining a male key portion adapted to mate with the female lock portion at the second end 112d of the cannula 110d, as shown in FIG. 25B. The male key portion 126d is therefore configured to mate with the female lock portion 112d when the stylet 120d is fully inserted into the cannula 110d, thus ensuring correct orientation of the first end 111d of the cannula relative to the first end 121d of the stylet for forming a cutting tip operable to penetrate bone and associated bone marrow. Furthermore, the rigid stylet 120d is configured to support the flexible tube 150d during an insertion procedure, and prevents deformation of the flexible tube until such deformation is desired, i.e., by removing the stylet from the tube before deformation is undertaken.

For example, during an insertion procedure, the hub 130d is engaged to the connector 140d such that the rigid stylet 120d is received within the cannula 110d and the flexible tube 150d to provide sufficient rigidity to the tube in order for the tube to withstand the force or torque applied thereto by a manual or powered driver. An example of a manual driver 200 is shown connected to the IO needle set 100d in FIG. 25C. Accordingly, the stylet 120d is configured to add stiffness to the flexible tube 150d during an insertion procedure so that the tube does not inadvertently bend out of shape due to the force and/or torque applied via the driver. In other words, the rigid stylet provides stability and rigidity to counteract the force and/or torque applied to the flexible tube as the penetrators are pushed and/or rotated by the driver to penetrate into dense bone.

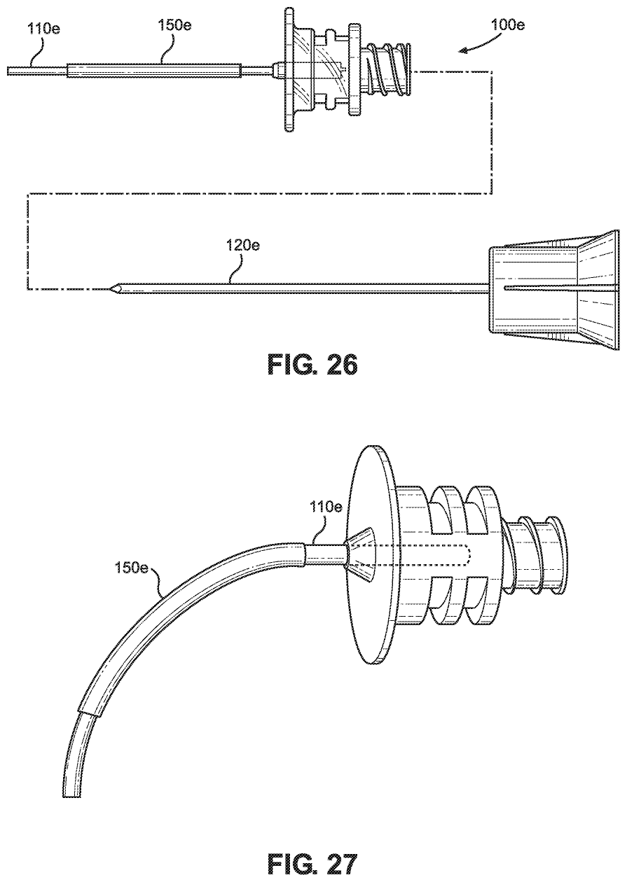

FIG. 26 depicts another implementation of a penetrator assembly, such as an IO needle set 100e. In particular, the IO needle set 100e comprises a flexible outer penetrator or cannula 110e configured to receive a rigid inner penetrator or stylet 120e. The cannula 110e comprises a hypo-tube which allows it to flex or bend similar to the other implementations of the flexible cannula previously described above. A flexible sleeve 150e, such as helical hollow strand tubing, is attached to an intermediate section of the hypo-tube 110e via soldering in order to prevent or reduce leakage of material passing through the hypo-tube. As shown in FIG. 27, when the rigid stylet is removed from the cannula, the hypo-tube 110e and the corresponding flexible sleeve 150e may be flexed to a bent configuration.

While the penetrator assembly having a flexible outer penetrator and a rigid inner penetrator has been described in terms of what may be considered to be specific aspects, the present disclosure is not limited to the disclosed aspects. Moreover, the many features and advantages of the disclosure are apparent from the detailed specification, and thus, it is intended by the appended claims to cover all such features and advantages of the disclosure which fall within the spirit and scope of the disclosure. Further, it is not desired to limit the disclosure to the exact construction and operation illustrated and described, and accordingly, all suitable modifications and equivalents may be resorted to, falling within the scope of the disclosure. Accordingly, the present disclosure should be considered as illustrative and not restrictive. As such, this disclosure is intended to cover various modifications and similar arrangements included within the spirit and scope of the claims, which should be accorded their broadest interpretation so as to encompass all such modifications and similar structures.

1. A penetrator assembly operable to provide access to an intraosseous space, the penetrator assembly comprising:
a flexible outer penetrator including a longitudinal bore and a distal end operable to penetrate bone and associated bone marrow;
a rigid inner penetrator including a distal end operable to penetrate bone and associated bone marrow;
a hub having a distal end connected to a proximal end of the flexible outer penetrator;
a connector having a distal end connected to a proximal end of the rigid inner penetrator and a proximal end configured to releasably engage a driver; and
the longitudinal bore of the flexible outer penetrator configured to removably receive the rigid inner penetrator to prevent or minimize the flexible outer penetrator from bending during an insertion procedure, and the flexible outer penetrator configured to bend after removal of the rigid inner penetrator from the longitudinal bore.

2. The penetrator assembly according to claim 1, further comprising a depth control collar connected to the outer penetrator adjacent to the distal end of the outer penetrator, the depth control collar comprising a distal end operable to contact the bone for preventing further penetration of the inner and outer penetrators into the intraosseous space.

3. The penetrator assembly according to claim 1, further comprising a distal cutting sleeve fixed to the distal end of the outer penetrator, the distal cutting sleeve operable to penetrate bone and associated bone marrow.

4. The penetrator assembly according to claim 1, wherein the flexible outer penetrator includes a plurality of slits operable to allow the outer penetrator to bend when the rigid inner penetrator is removed from the longitudinal bore of the flexible outer penetrator.

5. The penetrator assembly according to claim 4, wherein the plurality of slits are provided along an intermediate section of the flexible outer penetrator, the intermediate section located between the distal and proximal ends of the flexible outer penetrator.

6. The penetrator assembly according to claim 5, wherein the plurality of slits are arranged in a pattern along the length of the intermediate section of the flexible outer penetrator.

7. (canceled)

8. (canceled)

9. The penetrator assembly according to claim 4, wherein at least one of the plurality of slits is an elongated cut in a wall of the flexible outer penetrator.

10. The penetrator assembly according to claim 4, wherein at least one of the plurality of slits is a perforation in a wall of the flexible outer penetrator.

11. The penetrator assembly according to claim 4, wherein at least one of the plurality of slits is a notch or groove in a wall of the flexible outer penetrator.

12. (canceled)

13. (canceled)

14. (canceled)

15. The penetrator assembly according to claim 4, further comprising a covering sleeve attached to the flexible outer penetrator, the covering sleeve operable to prevent or minimize leakage of material from the flexible outer penetrator.

16. The penetrator assembly according to claim 15, wherein the covering sleeve is configured to cover the intermediate section of the flexible outer penetrator, the covering sleeve operable to prevent or minimize leakage of material from the plurality of slits.

17. The penetrator assembly according to claim 15, wherein the covering sleeve is operable to bend when bending the flexible outer penetrator.

18. The penetrator assembly according to claim 15, wherein the covering sleeve is operable to prevent or reduce leakage of material passing through the outer penetrator at a high pressure.

19-23. (canceled)

24. The penetrator assembly according to claim 15, wherein an outer diameter of the covering sleeve is less than or equal to the size of an outer diameter of the collar.

25. The penetrator assembly according to claim 15, wherein an outer diameter of the covering sleeve is less than or equal to the size of an outer diameter of the distal cutting sleeve.

26. The penetrator assembly according to claim 1, wherein the distal end of the connector is configured to releasably engage the proximal end of the hub.

27-33. (canceled)

34. A penetrator assembly operable to provide access to an intraosseous space, the penetrator assembly comprising:
a fluted drill tip operable to penetrate bone and associated bone marrow;
a flexible cannula including a longitudinal bore and a distal end connected to the fluted drill tip;
a rigid stylet configured to be releasably received within the longitudinal bore of the flexible cannula, the rigid stylet operable to prevent or minimize the flexible cannula from bending during an insertion procedure when the rigid stylet is received within the longitudinal bore of the flexible cannula, and the flexible cannula configured to bend after removal of the rigid stylet from the longitudinal bore;
a hub having a distal end connected to a proximal end of the flexible cannula; and
a connector having a distal end connected to a proximal end of the rigid stylet, and a proximal end configured to releasably engage a driver.

35. The penetrator assembly according to claim 34, wherein the fluted drill tip comprises a head portion, a body portion, and a cutting flute extending along both the head and body portions, the cutting flute defining a channel operable to allows for blood and/or bone marrow samples to be aspirated from the intraosseous space or for medication to be delivered to the intraosseous space.

36. The penetrator assembly according to claim 34, wherein the flexible cannula includes a plurality of slits operable to allow the cannula to bend when the rigid stylet is removed from the longitudinal bore of the cannula.

37-46. (canceled)

47. The penetrator assembly according to claim 36, further comprising a covering sleeve attached to the flexible cannula, the covering sleeve operable to prevent or minimize leakage of material from the flexible cannula.

48-66. (canceled)
